(19) United States
(12) Patent Application Publication
(10) Pub. No.: US 2009/0099036 A1
(43) Pub. Date: Apr. 16, 2009

(54) METHODS AND COMPOSITIONS FOR SCREENING GLYCAN STRUCTURES

(75) Inventors: David M. Lubman, Ann Arbor, MI (US); Tasneem H. Patwa, Ann Arbor, MI (US); Jia Zhao, Ann Arbor, MI (US)

Correspondence Address: Casimir Jones, S.C., 440 Science Drive, Suite 203, Madison, WI, 53711, US

(73) Assignee: THE REGENTS OF THE UNIVERSITY OF MICHIGAN, Ann Arbor, MI (US)

(21) Appl. No.: 12/297,384

(22) PCT Filed: Apr. 18, 2007
(86) PCT No.: PCT/US07/09480

§ 371 Date: Oct. 16, 2008

Related U.S. Application Data
(60) Provisional application No. 60/792,745, filed on Apr. 18, 2006.

(51) Int. Cl. C40B 30/04; C40B 40/10

(52) U.S. Cl. 506/9; 506/18

(57) ABSTRACT

The present invention relates to methods and compositions for screening of glycan structures. In particular, the present invention provides methods and compositions for global profiling of glycoprotein states by utilizing a glycoprotein microarray format.

The present invention further provides for methods and compositions of glycoprotein microarray formats for differentiating between different glycosylation states associated with disease states.

This invention was made with government support under R01 CA106402 and R01 GM49500 awarded by the National Institute of Health. The government has certain rights in the invention.

### FIELD OF THE INVENTION

The present invention relates to methods and compositions for screening of glycan structures. In particular, the present invention provides methods and compositions for global profiling of glycoprotein states by utilizing a glycoprotein microarray format.

The present invention further provides for methods and compositions of glycoprotein microarray formats for differentiating between different glycosylation states associated with disease states.

### BACKGROUND OF THE INVENTION

Glycoproteins are proteins that have glycans (polysaccharides), or sugar molecules, attached to them through a process known as glycosylation. Glycoproteins are the most diverse group of modifications known in proteins, and variants of glycoproteins (glycoforms) can lead to changes in protein activity or function that may lead to disease. Many clinical biomarkers and therapeutic targets in cancer are glycoproteins (Dube et al., 2005, Nat. Rev. Drug Disc. 4:477-488; Orntoft et al., 1999, Electrophoersis 20:362-371; Semmes et al., 2006, J. Cell Biochem. Epub.), such as CA125 in ovarian cancer, Her2/neu in breast cancer, and prostate-specific antigen (PSA) in prostate cancer. In addition, alterations in protein glycosylation have been correlated with the development of cancer and other disease states (Block et al., 2005, Proc. Natl. Acad. Sci. 102:779-784). Global screening of glycoprotein profiles in varied biological states (e.g., different stages of cancer, etc.) can provide valuable information regarding key pathways in disease states useful for drug discovery and disease therapeutic applications.

Increased interest in glycoproteomes has sparked related research in the microarray field. A majority of efforts have focused on carbohydrate microarrays (Nimrichter et al., 2004, Glycobiology 14:197-203; Wang and Lu, 2004, Physiol. Genomics 18:245-248; Feizi et al., 2003, Curr. Opin. Struct. Biol. 13:637-645). Such studies are critical in assessing antibody specificity to glycans and determining currently uncharacterized glycosylation structures that elicit responses in cells. However, oligosaccharides are difficult to synthesize, there are limited availability of enzymes for alternate synthesis strategies, and there are problems with purification when isolating naturally occurring oligosaccharides. Furthermore, the low mass and hydrophilic nature of most oligosaccharides makes non-covalent immobilization difficult for some glycans (Wang and Lu, 2004), with this problem being overcome somewhat by covalent attachment of glycans to solid surfaces using film-coated photoactivable surfaces (Angeloni et al., 2005, Glycobiol. 15:31-41) and array coupling via flexible linker molecules (Schwarz et al., 2003, Glycobiol. 13:749-754). Although carbohydrate arrays provide valuable information about carbohydrate-interacting proteins, they do not allow one to directly study changes in glycosylation in real biological systems.

Current technologies for glycan analysis such as mass spectrometry (Wang et al., 2006, Glycobiol. Epub.), lectin affinity chromatography (Qiu et al., 2005, Anal. Chem. 77:2802-2809; Qiu et al., 2005, Anal Chem. 77:7225-7231) and western blotting are time consuming and some, such as mass spectrometry, require expertise and are technically difficult (Novotny et al., 2005, J. Sep. Sci. 28:1956-1968). Studies using lectin arrays have focused on assessing specificity of arrayed lectins (Kuno et al., 2005, Nat. Methods 2:851-856; Pilobello et al., 2005, Chembiochem. 6:985-989) as well as changes in lectin binding of whole cell lysates that have undergone a treatment of some kind (Angeloni et al., 2005). However, lectin array platforms do not allow the screening of whole glycoproteomes in a way that will enable the study of both changes in overall glycoprotein patterns as well as changes in an individual protein's glycan expression within that glycoproteome.

What are needed are improved methods and compositions for the identification of glycoproteins and their variant glycoforms and glycan structures to advance the diagnosis, management, and research surrounding human diseases and disorders. High throughput methods and compositions that assess a diverse range of glycosylation states would provide valuable information for drug discovery and disease therapeutics, and provide valuable tools for ongoing research.

### SUMMARY OF THE INVENTION

The present invention relates to methods and compositions for screening of glycan structures. In particular, the present invention provides methods and compositions for global profiling of glycoprotein states by utilizing a glycoprotein microarray format. The present invention further provides the method of glycoprotein microarray formats for differentiating between different glycosylation states associated with cancer.

Glycoproteins are the most heterogeneous group of modifications known in proteins. Glycans show a high structural diversity reflecting inherent functional diversity. N- and O-oligosaccharide variants on glycoproteins (glycoforms) can lead to alterations in protein activity or function that may manifest itself as overt disease (Rudd et al., 2001, Science 291:2370-2376; Kobata et al., 2005, Immunol. Cell Biol. 83:429-439). In addition, the alteration in protein glycosylation which occurs through varying the heterogeneity of glycosylation sites or changing glycan structure of proteins on the cell surface and in body fluids have been shown to correlate with the development of cancer and other disease states (Block et al., 2005). Identification of glycoprotein glycoforms is becoming increasingly important to the diagnosis and management of human diseases as more diseases are found to result from glycan structural alterations such as I-cell disease, and congenital disorders of glycosylation leukocyte adhesion deficiency type II (Durand and Seta, 2000, Clin. Chem. 46:795-805).

There are approximately 100 human glycan-binding proteins (i.e. lectins) according to genomic analysis. The variety of lectin protein folds suggests that there may be additional lectin groups not yet discovered (Nimrichter et al., 2004; Drickamer et al., 2002, Genome Biol. 3:1034).

Protein microarrays have proven to be useful as a high-throughput screening method for whole cell lysates, fractionated proteomes, tissues and antigen-antibody reactions (Templin et al., 2003, Proteomics 3:2155-2166; Pal et al., 2006, Anal. Chem. 78:702-710; Yan et al., 2003, Proteomics 3:1228-1235; Orchekowski et al., 2005, Cancer Res. 65:11193-11202).

Pancreatic cancer is the most frequent adenocarcinoma and has the worst prognosis of all cancers, with a five-year survival rate of <3 percent, accounting for the 4^{th} largest number of cancer deaths in the USA (Jemal et al., CA Cancer J. Clin., 53: 5-26, 2003). Pancreatic cancer occurs with a frequency of around 9 patients per 100,000 individuals making it the 11^{th} most common cancer in the USA. Currently the only curative treatment for pancreatic cancer is surgery, but only ˜10-20% of patients are candidates for surgery at the time of presentation, and of this group, only ˜20% of patients who undergo a curative operation are alive after five years (Yeo et al., Ann. Surg., 226: 248-257, 1997; Hawes et al., Am. J. Gastroenterol., 95: 17-31, 2000).

The poor prognosis and lack of effective treatments for pancreatic cancer arise from several causes. Pancreatic cancer tends to rapidly invade surrounding structures and undergo early metastatic spreading, such that it is the cancer least likely to be confined to its organ of origin at the time of diagnosis (Greenlee et al., 2001. CA Cancer J. Clin., 51: 15-36, 2001). Finally, pancreatic cancer is highly resistant to both chemo- and radiation therapies (Greenlee et al., supra). Currently the molecular basis for these characteristics of pancreatic cancer is unknown.

Therefore, one embodiment of the present invention describes a strategy that utilizes natural glycoprotein microarrays with a lectin detection format to study individual glycoprotein profiles of different biological states. The strategy employs a liquid fractionated protein microarray approach to screen all glycoproteins in a complex sample on a single array. In some embodiments, glycoproteins are first enriched on a general lectin column and then separated by non-porous reverse-phase HPLC (NP RP-HPLC). The separated proteins are subsequently spotted on nitrocellulose slides or other desired support and probed with lectins demonstrating different glycan structural binding specificities. In some embodiments, the glycoprotein-lectin interaction is assessed using a biotin-streptavidin system or similar systems. This method allows for profiling the distribution of glycans in the human glycoproteome, and also allows the study of changes in glycan expression on a global scale and on individual glycoproteins, since each glycoprotein sample is a unique spot on the array. In some embodiments a glycosylation based microarray can be used to study and to differentiate between different stages of disease (e.g., pancreatic) and cancer (e.g., pancreatic) as compared to normal tissue, in the hope of furnishing drug discovery and therapeutic treatment alternatives for diseases such as cancers and for diagnostic purposes.

Methods and compositions of the glycosylation microarray format are not intended to be limited to the study of and differentiation of diseases alone, as one skilled in the art would recognize that the methods and compositions of the present invention are applicable to any condition or biological state where glycosylation and/or glycan structural states provide relevant information, as well as use of the present methods and compositions to study differences in any protein's glycosylation and/or glycan structural state.

One embodiment of the present invention is a method for high throughput determination of glycan structures comprising providing a sample comprising a glycoproteome of a biological sample, providing a solid support, applying said sample to said solid support such that discrete areas containing said sample are created on said solid support, providing one or more lectins, contacting said one or more lectins with said solid support containing said discrete areas containing said sample, and determining the glycan structure of glycoproteins in said glycoproteome by the binding of said one or more lectins to said discrete areas on said solid support containing said sample.

In some embodiments, determining the glycan structure of glycoproteins in said glycoproteome further comprises determining the presence or absence of cancer. In some embodiments, the cancer being determined is pancreatic cancer. In some embodiments, the one or more lectins are bound to a first binding member, which, for example, is biotin. In some embodiments, the first binding member is further bound to a second binding member (e.g., a fluorescent moiety, streptavidin, etc.). In some embodiments, a second binding member is further associated with a fluorescent moiety. In some embodiments, the glycoproteome sample is derived from a serum sample. In some embodiments, the serum sample is from a subject suffering from diseases of the pancreas (e.g., pancreatitis, cancer), and/or could be from a subject that does not have such diseases. In some embodiments, the sample of the present invention can be purified by lectin chromatography, and/or further purified using non-porous reverse phase HPLC. In some embodiments, there are two or more lectins used to screen the sample. In some embodiments, there are three or more lectins used to screen the sample. In some embodiments, there are four or more lectins used to screen the sample. In some embodiments, lectins used to screen the glycoproteome include, but are not limited to Concanavalin A, Maaackia amurensis II, Aleuria aurantia, Sambucus nigra bark, and Peanut agglutinin.

In one embodiment, the present invention includes compositions comprising a solid support comprising discrete areas upon which are affixed purified or partially purified glycoproteins, one or more lectins wherein a lectin recognizes a different glycan structure, and a compound which binds to said lectin either directly or indirectly. In some embodiments, the lectins of the composition are conjugated to a first member of a binding pair. In some embodiments, the first binding pair is a biotin. In some embodiments, the compound that binds directly to a lectin is a fluorescently labeled antibody. In some embodiments, the compounds that binds indirectly to a lectin is a fluorescently labeled straptavidin molecule.

All references listed are incorporated herein in their entireties.

### DESCRIPTION OF THE FIGURES

FIG. 1 shows an experimental strategy for studying glycoproteins in an embodiment of the present invention; 1) lectin purification, 2) non-porous RP-HPLC separation and fraction collection, 3) microarray production, 4) lectin detection using biotin-streptavidin-AlexaFluor®555, and 5) image acquisition and analysis.

FIG. 2 shows the images of printed glycoprotein standards probed with different lectins. Each bracket on the right represents the dilution series (2.0-0.025 mg/ml) of the indicated glycoprotein standard (n=9).

FIG. 3 shows the linearity in the response of the glycoprotein standards to the lectin probes; a) glycan distribution (y axis, as fluorescence) on the specific glycoprotein (x axis), b) Ribonuclease B with ConA lectin probe, c) thyroglobulin with AAL lectin probe, d) transferrin with SNA lectin probe, e) fetuin with MAL lectin probe, and f) asialofetuin with PNA lectin probe.

FIG. 4 shows the differences in glycosylation from sera of different biological states; a) reverse phase chromatogram of enriched glycoproteins from normal and pancreatitis sera, b) left arrow fluorescence microarray data, and c) right arrow fluorescence microarray data, with corresponding bar graph (x-axis is lectin probe, y-axis is relative fluorescence).

FIG. 5 shows the comparison of differential glycosylation patterns in normal vs. pancreatic cancer serum samples in microarray format and with corresponding bar graph (x-axis is lectin probe, y-axis is relative fluorescence); a) differential glycan expression of human anti-thrombinIII precursor (ATIII), b) differential glycan expression of human leucine rich α-2-glycoprotein precursor (LRG), c), differential glycan expression of human α-2-macroglobulin precursor (alpha-2-M) and d) differential glycan expression of human complement precursors C3 and C4.

### DEFINITIONS

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include tissues and blood products, such as plasma, serum and the like. Such examples are not however to be construed as limiting the sample types applicable to the present invention.

As used herein, the term “peptide” refers to a compound comprising from two or more amino acid residues wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g. solid phase synthesis) or molecular biology techniques (see Sambrook, J. et al., Molecular Cloning: A Laboratory Manual, Cold Spring Harbor Press, Cold Spring Harbor, N.Y. (1989)).

As used herein, the term “peptidomimetic” refers to molecules which are not polypeptides, but which mimic aspects of their structures. For example, polysaccharides can be prepared that have the same functional groups as peptides. A peptidomimetic comprises at least two components, the binding moiety or moieties, and the backbone or supporting structure.

As used herein, the term “antibody” encompasses both monoclonal and polyclonal full-length antibodies and functional fragments thereof (e.g. maintenance of binding to target molecule). Antibodies can include those that are chimeric, humanized, primatized, veneered or single chain antibodies.

As used herein, the terms “agent”, “compound” or “drug” are used to denote a compound or mixture of chemical compounds, a biological macromolecule such as an antibody, a nucleic acid, or an extract made from biological materials such as bacteria, plants, fungi, or animal (particularly mammalian) cells or tissues that are suspected of having therapeutic properties. The compound, agent or drug may be purified, substantially purified or partially purified.

As used herein, the term “fragment” when in reference to a protein (e.g. “a fragment of a given protein”) refers to portions of that protein. The fragments may range in size from two amino acid residues to the entire amino acid sequence minus one amino acid. In one embodiment, the present invention contemplates “functional fragments” of a protein. Such fragments are “functional” if they can bind with their intended target protein (e.g. the functional fragment may lack the activity of the full length protein, but binding between the functional fragment and the target protein is maintained).

As used herein, the term “antagonist” refers to molecules or compounds (either native or synthetic) that inhibit the action of a compound (e.g., receptor channel, ligand, etc.). Antagonists may or may not be homologous to these compounds in respect to conformation, charge or other characteristics. Thus, antagonists may be recognized by the same or different receptors that are recognized by an agonist. Antagonists may have allosteric effects that prevent the action of an agonist. Or, antagonists may prevent the function of the agonist.

As used herein, the term “subject” refers to any biological entity that can be used for experimental work. For example, a “subject” can be a mammal such as a mouse, rat, pig, dog, and non-human primate. Preferably the subject is a human.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental exposure, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

As used herein, the terms “anticancer agent” and “anticancer drug” refer to any therapeutic agents (e.g., chemotherapeutic compounds and/or molecular therapeutic compounds), radiation therapies, or surgical interventions, used in the treatment of hyperproliferative diseases such as cancer (e.g., in mammals).

As used herein “test compound” refers to any chemical entity, pharmaceutical, drug, and the like that can be used to treat or prevent a disease, illness, sickness, or disorder of bodily function. Test compounds comprise both known and potential therapeutic compounds. A test compound can be determined to be therapeutic by screening, using the screening methods of the present invention. A known therapeutic compound refers to a therapeutic compound that has been shown (e.g., through animal trial or prior experience with administration to humans) to be effective in such treatment or prevention.

As used herein, the term “chemotherapeutic agent” refers to any compound, drug, or agent used to treat various forms of cancer. Chemotherapeutic agents have the ability inhibit cancer cell growth and/or kill cancer cells. Chemotherapeutic agents to be used in conjunction with the compounds of the present invention, include but are not limited to, estrogen receptor blockers, estrogen blockers, and additional oncolytic compounds, drugs and agents as described herein.

As used herein, the term “multiphase protein separation” refers to protein separation comprising at least two separation steps. In some embodiments, multiphase protein separation refers to two or more separation steps that separate proteins based on different physical properties of the protein (e.g., a first step that separates based on protein charge and a second step that separates based on protein hydrophobicity).

As used herein, the term “protein profile maps” refers to representations of the protein content of a sample. For example, “protein profile map” includes 2-dimensional displays of total protein expressed in a given cell. In some embodiments, protein profile maps may also display subsets of total protein in a cell. Protein profile maps may be used for comparing “protein expression patterns” (e.g., the amount and identity of proteins expressed in a sample) between two or more samples. Such comparisons find use, for example, in identifying proteins that are present in one sample (e.g., a cancer cell) and not in another (e.g., normal tissue), or are over- or under-expressed in one sample compared to the other.

As used herein, the term “2-dimensional protein map” refers to a “protein profile map” that represents (e.g., on two axis of a graph) two properties of the protein content of a sample (e.g., including but not limited to, hydrophobicity and isoelectric point).

As used herein the term “differential display map” and equivalents “differential display plot” and “differential display image” refer to a “protein profile map” that shows the subtraction of one protein profile map from another protein profile map. A differential display map thus shows the differences in proteins present between two samples. A differential display image may also show differences in the abundance of a protein between the two samples. In some embodiments, multiple colors or color gradients are used to represent proteins from each of the two samples.

As used herein, the term “separating apparatus capable of separating proteins based on a physical property” refers to compositions or systems capable of separating proteins (e.g., at least one protein) from one another based on differences in a physical property between proteins present in a sample containing two or more protein species. For example, a variety of protein separation columns and compositions are contemplated including, but not limited to ion exclusion, ion exchange, normal/reversed phase partition, size exclusion, ligand exchange, liquid/gel phase isoelectric focusing, affinity chromatography and adsorption chromatography. These and other apparatuses are capable of separating proteins from one another based on their size, charge, hydrophobicity, and ligand binding affinity, among other properties. A “liquid phase” separating apparatus is a separating apparatus that utilizes protein samples contained in liquid solution, wherein proteins remain solubilized in liquid phase during separation and wherein the product (e.g., fractions) collected from the apparatus are in the liquid phase. This is in contrast to gel electrophoresis apparatuses, wherein the proteins enter into a gel phase during separation. Liquid phase proteins are much more amenable to recovery/extraction of proteins as compared to gel phase. In some embodiments, liquid phase proteins samples may be used in multi-step (e.g., multiple separation and characterization steps) processes without the need to alter the sample prior to treatment in each subsequent step (e.g., without the need for recovery/extraction and resolubilization of proteins).

As used herein, the term “displaying proteins” refers to a variety of techniques used to interpret the presence of proteins within a protein sample. Displaying includes, but is not limited to, visualizing proteins on a computer display representation, diagram, autoradiographic film, list, table, chart, etc. “Displaying proteins under conditions that first and second physical properties are revealed” refers to displaying proteins (e.g., proteins, or a subset of proteins obtained from a separating apparatus) such that at least two different physical properties of each displayed protein are revealed or detectable. For example, such displays include, but are not limited to, tables including columns describing (e.g., quantitating) the first and second physical property of each protein and two-dimensional displays where each protein is represented by an X,Y locations where the X and Y coordinates are defined by the first and second physical properties, respectively, or vice versa. Such displays also include multi-dimensional displays (e.g., three dimensional displays) that include additional physical properties. In some embodiments, displays are generated by “display software.”

As used herein, “characterizing protein samples under conditions such that first and second physical properties are analyzed” refers to the characterization of two or more proteins, wherein two different physical properties are assigned to each analyzed (e.g., displayed, computed, etc.) protein and wherein a result of the characterization is the categorization (i.e., grouping and/or distinguishing) of the proteins based on these two different physical properties. For example, in some embodiments, two proteins are separated based on isoelectric point and hydrophobicity.

As used herein, the term “comparing first and second physical properties of separated protein samples” refers to the comparison of two or more protein samples (or individual proteins) based on two different physical properties of the proteins within each protein sample. Such comparing includes grouping of proteins in the samples based on the two physical properties and comparing certain groups based on just one of the two physical properties (i.e., the grouping incorporates a comparison of the other physical property).

As used herein, the term “delivery apparatus capable of receiving a separated protein from a separating apparatus” refers to any apparatus (e.g., microtube, trough, chamber, etc.) that receives one or more fractions or protein samples from a protein separating apparatus and delivers them to another apparatus (e.g., another protein separation apparatus, a reaction chamber, a mass spectrometry apparatus, etc.).

As used herein, the term “detection system capable of detecting proteins” refers to any detection apparatus, assay, or system that detects proteins derived from a protein separating apparatus (e.g., proteins in one or more fractions collected from a separating apparatus). Such detection systems may detect properties of the protein itself (e.g., UV spectroscopy) or may detect labels (e.g., fluorescent labels) or other detectable signals associated with the protein. The detection system converts the detected criteria (e.g., absorbance, fluorescence, luminescence etc.) of the protein into a signal that can be processed or stored electronically or through similar means (e.g., detected through the use of a photomultiplier tube or similar system).

As used herein, the term “buffer compatible with an apparatus” and “buffer compatible with mass spectrometry” refer to buffers that are suitable for use in such apparatuses (e.g., protein separation apparatuses) and techniques. A buffer is suitable where the reaction that occurs in the presence of the buffer produces a result consistent with the intended purpose of the apparatus or method. For example, a buffer compatible with a protein separation apparatus solubilizes the protein and allows proteins to be separated and collected from the apparatus. A buffer compatible with mass spectrometry is a buffer that solubilizes the protein or protein fragment and allows for the detection of ions following mass spectrometry. A suitable buffer does not substantially interfere with the apparatus or method so as to prevent its intended purpose and result (i.e., some interference may be allowed).

As used herein, the term “automated sample handling device” refers to any device capable of transporting a sample (e.g., a separated or un-separated protein sample) between components (e.g., separating apparatus) of an automated method or system (e.g., an automated protein characterization system). An automated sample handling device may comprise physical means for transporting sample (e.g., multiple lines of tubing connected to a multi-channel valve). In some embodiments, an automated sample handling device is connected to a centralized control network. In some embodiments, the automated sample handling device is a robotic device.

As used herein, the term “switchable multi channel valve” refers to a valve that directs the flow of liquid through an automated sample handling device. The valve preferably has a plurality of channels (e.g., 2 or more, and preferably 4 or more, and more preferably, 6 or more). In addition, in some embodiments, flow to individual channels is “switched” on and off. In some embodiments, valve switching is controlled by a centralized control system. A switchable multi-channel valve allows multiple apparatus to be connected to one automated sample handler. For example, sample can first be directed through one apparatus of a system (e.g., a first chromatography apparatus). The sample can then be directed through a different channel of the valve to a second apparatus (e.g., a second chromatography apparatus).

As used herein, the terms “centralized control system” or “centralized control network” refer to information and equipment management systems (e.g., a computer processor and computer memory) operably linked to multiple devices or apparatus (e.g., automated sample handling devices and separating apparatus). In preferred embodiments, the centralized control network is configured to control the operations or the apparatus and devices linked to the network. For example, in some embodiments, the centralized control network controls the operation of multiple chromatography apparatus, the transfer of sample between the apparatus, and the analysis and presentation of data.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refers to a device that is able to read a program from a computer memory (e.g., ROM or other computer memory) and perform a set of steps according to the program.

As used herein, the term “hyperlink” refers to a navigational link from one document to another, or from one portion (or component) of a document to another. Typically, a hyperlink is displayed as a highlighted word or phrase that can be selected by clicking on it using a mouse to jump to the associated document or documented portion.

As used herein, the term “display screen” refers to a screen (e.g., a computer monitor) for the visual display of computer generated images. Images are generally displayed by the display screen as a plurality of pixels.

As used herein, the term “computer system” refers to a system comprising a computer processor, computer memory, and a display screen in operable combination. Computer systems may also include computer software.

As used herein, the term “directly feeding” a protein sample from one apparatus to another apparatus refers to the passage of proteins from the first apparatus to the second apparatus without any intervening processing steps. In such a case, the second apparatus “directly receives” the protein sample from the first apparatus. For example, a protein that is directly fed from a protein separating apparatus to a mass spectrometry apparatus does not undergo any intervening digestion steps (i.e., the protein received by the mass spectrometry apparatus is undigested protein).

As used herein, “purified and partially purified” relate to proteins which have been separated by some extent from their native environment. For example, the present invention relates to glycoproteins which have been partially purified by applying a complex biological sample to a lectin column. The glycoprotein sample from the lectin column is then purified to a greater extent by using non-porous reverse phase HPLC.

As used herein, the terms “solid support” or “support” refer to any material that provides a solid or semi-solid structure with which another material can be attached. Such materials include smooth supports (e.g., metal, glass, plastic, silicon, and ceramic surfaces) as well as textured and porous materials. Such materials also include, but are not limited to, gels, rubbers, polymers, dendrimers and other non-rigid materials. Solid supports need not be flat. Supports include any type of shape including spherical shapes (e.g., beads). Materials attached to solid support may be attached to any portion of the solid support (e.g., may be attached to an interior portion of a porous solid support material). Preferred embodiments of the present invention have biological molecules such as proteins attached to solid supports. A biological material is “attached” to a solid support when it is associated with the solid support through a non-random chemical or physical interaction. In some preferred embodiments, the attachment is through a covalent bond. However, attachments need not be covalent or permanent. In some embodiments, materials are attached to a solid support through a “spacer molecule” or “linker group.” Such spacer molecules are molecules that have a first portion that attaches to the biological material and a second portion that attaches to the solid support. Thus, when attached to the solid support, the spacer molecule separates the solid support and the biological materials, but is attached to both.

As used herein, the term “microarray” refers to a solid support with a plurality of molecules (e.g., proteins) bound to its surface. Additionally, the term “patterned microarrays” refers to microarray substrates with a plurality of molecules non-randomly bound to its surface.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxygenin; luminogenic, phosphorescent or fluorogenic moieties; mass tags; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, characteristics of mass or behavior affected by mass (e.g., MALDI time-of-flight mass spectrometry), and the like. A label may be a charged moiety (positive or negative charge) or alternatively, may be charge neutral. Labels can include or consist of nucleic acid or protein sequence, so long as the sequence comprising the label is detectable.

The term “epitope” as used herein refers to that portion of an antigen that makes contact with a particular antibody.

### DETAILED DESCRIPTION OF THE INVENTION

The present invention relates to methods and compositions for screening of glycan structures. In particular, the present invention provides methods and compositions for global profiling of glycoprotein states by utilizing a glycoprotein microarray format.

The present invention further provides the method of glycoprotein microarray formats for differentiating between different glycosylation states associated with cancer.

Protein glycosylation has been implicated in key biological processes including, for example, immunological recognition, cellular adhesion, protein folding and signaling, as well as disease progression. Glycan structures on proteins are highly diverse, and different forms and variant of attachment of glycans on proteins alter the protein's function, oftentimes resulting in different disease states as previously described. For example, glycosylation is relevant to many cancers, including pancreatic cancer.

Pancreatic cancer is a major oncologic challenge and cellular events that allow for measurable early detection are desperately needed. There is currently great interest in developing protein-based serum markers for cancer. Based on the inaccessible location of the pancreas, a serum test is needed to screen patients for the early detection of this disease, particularly in high-risk populations. An important target for serum detection involves the presence of glycosylated proteins. Protein glycosylation has long been recognized as a very common post-translational modification, playing a fundamental role in many biological processes such as immune response and cellular regulation (Bertozzi et al., Science 2001, 291, 2357-2364; Rudd P M et al., 2001). The glycoproteome is one of the major subproteomes of human serum, where glycoproteins secreted into the blood stream comprise a major part of the serum proteome (Anderson et al., Electrophoresis 1998, 19, 1853-1861). Many clinical biomarkers and therapeutic targets in cancer are glycoproteins, such as CA125 in ovarian cancer, Her2/neu in breast cancer and prostate-specific antigen in prostate cancer. In addition, the alteration in protein glycosylation which occurs through varying the heterogeneity of glycosylation sites or changing glycan structure of proteins on the cell surface and in body fluids have been shown to correlate with the development of cancer and other disease states (Durand et al., Chem 2000, 46, 795-805). Therefore, a method that can (1) quantitatively analyze glycoprotein abundance and (2) detect the extent of glycosylation alteration and the carbohydrate structure that correlate with pancreatic cancer will be useful for the discovery of new potential diagnostic markers of this disease.

Sialic acids are generally found in the non-reducing terminus of most glycoproteins and glycolipids via a α-2,3 or α-2,6 linkage to galactose or Hex-NAc. Sialic acids are important regulators of cellular and molecular interactions. They can either mask recognition sites or serve as recognition determinants (Kelm et al., Int Rev Cytol 1997, 175, 137-240). Increased sialylation of tumor cell surfaces is well known and is due to either increased activity of the sialyltransferases or due to the increased branching of N-linked carbohydrates leading to termini which can be sialylated (Orntoft et al., Electrophoresis 1999, 20, 362-371). Aberrant sialylation in cancer cells is thought to be a characteristic feature associated with malignant properties including invasiveness and metastatic potential.

Various methods have been developed to enrich glycoproteins. Zhang et al. have developed a method to enrich glycoproteins through hydrazide chemistry (Zhang et al., Nat Biotechnol 2003, 21, 660-666). In this method, the captured glycopeptides were deglycosylated by PNGase F and quantified by isotope labeling. Lectin affinity chromatography has recently been widely used to purify glycoproteins with specific structures. Hancock and coworkers developed a multi-lectin affinity column, which combines ConA, WGA and Jacalin to capture the majority of glycoproteins present in human serum (Yang et al., J Chromatogr A 2004, 1053, 79-88). In related work, Regnier et al utilized serial lectin affinity chromatography (SLAC) for fractionation and comparison of glycan site heterogeneity on glycoproteins derived from human serum (Qiu et al., Anal Chem 2005, 77, 7225-7231; Qiu et al., Anal Chem 2005, 77, 2802-2809). Novotny et al combined silica based lectin microcolumns with high-resolution separation techniques for enrichment of glycoproteins and glycopeptides (Madera et al., Anal Chem 2005, 77, 4081-4090).

In some embodiments, to illustrate the systems and methods of the present invention, experiments conducted during the course of development of the present invention analyzed pancreatic cancer serum using lectin affinity chromatography followed by fractionation using RP-HPLC, the fractions of which were spotted on slides as microarrays and probed with different biotin labeled lectins which recognized different glycan structures. The method was used to identify glycan structures specific to different pancreatic disease states, such as pancreatitis and pancreatic cancer. The expression of glycoproteins with different sub-structures were compared between normal, pancreatitis, and pancreatic cancer serum based on the bound lectin. Altered glycoproteins were digested and identified by LC-MS/MS. The structures of the released carbohydrate from purified serum proteins were studied using a MALDI-quadrupole-ion trap T of (MALDI-QIT) mass spectrometer. This method was used to detect the change of the isoforms and extent of glycosylation of target glycoproteins in the different sera. Glyco-peptide mapping was performed using LC-ESI-TOF MS to study the difference of glycosylation efficiency on the glycosylation site of proteins between normal and pancreatic disease sera. This approach allows for glycan expression in the same protein to be evaluated in normal versus disease state samples. Therefore, the methods and compositions of the present invention can quantitatively analyze both glycoprotein abundance and carbohydrate structural changes, and correlated those changes in biolological systems, for example, using the present methods and compositions to determine glycan changes associated with any desired biological state. By screening the glycoproteome, patterns of glycoprotein abundance and/or carbohydrate structural changes across multiple different proteins can be analyzed to provide a rich source of information for diagnostic, research, and therapeutic applications.

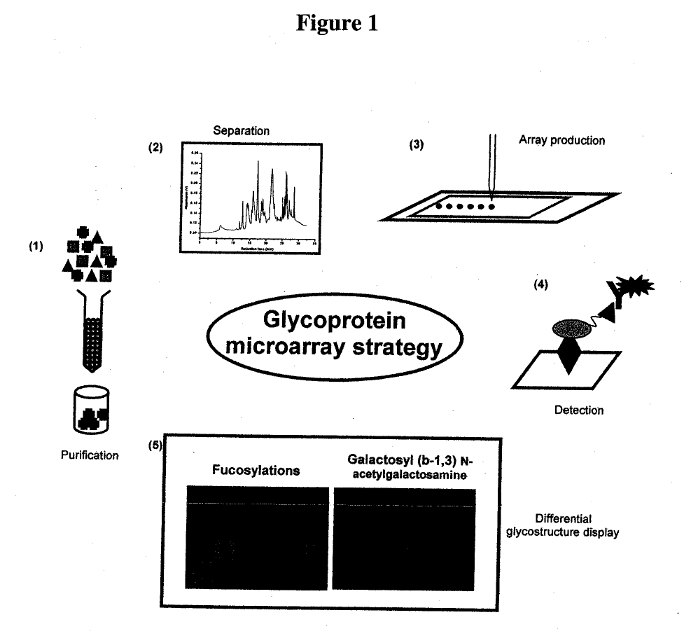

In one embodiment, the present invention provides a purification method for glycoproteins (e.g., lectin chromatography) found in complex biological samples (e.g., samples that contain one or more protein or other biological components such as serum, whole cell lysates, etc). In some embodiments, the purified glycoproteins are further separated and fractionated utilizing reverse phase HPLC. In some embodiments, the separated and fractionated glycoproteins are spotted onto a slide or other solid support surface that allows for high throughput screening of the glycoproteins. For example, the glycoprotein fractions are spotted onto a slide (e.g. nitrocellulose, glass, etc.) wherein each spot contains, for example, from 0.1 ng-3 μg of each glycoprotein fraction. In some embodiments, the glycoprotein fractions that are spotted onto a slide are approximately 450 μm in diameter and spaced approximately 600 μm apart, although the present invention is not limited by the dimensions used. It is contemplated that the present invention is not limited to the type of slide used and the procedure used for spotting the proteins onto the slide. For example, the following United States patents describe methods and compositions for creating slides for protein microarrays, and they are incorporated herein in their entireties; U.S. Pat. Nos. 6,936,311, 6,699,665, 6,528,291, 6,815,078, 6,733,894, 6,426,183, 6,403,368, 5,501,986, 6,929,944, 6,246,833, 5,028,657, 6,594,432, 6,953,551. In some embodiments, the spotted glycoprotein fractions are further allowed to dry on the microarray slide. In some embodiments, the slides are further contacted with a labeled (e.g., biotin, fluorescent, luminescent, etc.) lectin. In some embodiments, the labeled lectin is specific for a particular glycan structure as described, for example, in Table 1. In some embodiments, the microarray slide containing the glycoprotein fractions that have been contacted with a labeled lectin, are further contacted with a secondary compound that recognizes the label (e.g., streptavidin, antibody, etc.) and binds to it. For example, for a biotinylated lectin, a streptavidin molecule is used as a secondary labeling compound. In some embodiments, the secondary compound is additionally labeled with a detectable moiety (e.g., a fluorescent moiety, or luminescent moiety, etc.). In some embodiments, the secondary label is detected by detectable means (e.g., fluorometer, luminometer, etc.). For example, if the secondary label is streptavidin that has been labeled with a fluorophore, then a fluorometer would be used to detect the fluorescence of the fluorophore. In some embodiments, the detectable signal (e.g., fluorescence, luminescence, etc.) is then correlated to a disease state or stage in a sample when compared to an appropriate normal (i.e., known non-disease state or stage) sample. A pictorial representation of one embodiment of the present invention can be seen in FIG. 1.

TABLE 1
Biotinylated lectins used for glycan
detection and their specificities

| Biotinylated Lectin | Glycan structure detected |
|---|---|
| Concanavilin A (ConA) | a-linked mannose |
| Maackia Amurensis II (MAL) | sialic acid in an (a-2,3) linkage |
| Aleuria Aurantia (AAL) | fucose linked (a-1,6) to N- |
| | acetylglucosamine or to fucose linked |
| | (a-1,3) to N-acetyllactosamine |
| Sambucus Nigra (Elderberry) | sialic acid attached to terminal |
| bark (SNA) | galactose in (a-2,6), and to a lesser |
| | degree, (a-2,3), linkage |
| Peanut Agglutinin (PNA) | galactosyl (b-1,3) N-acetylgalactosamine |

In some embodiments, the present invention provides a multi-phase separation method (e.g., a lectin chromatography preceded by or followed by additional chromatography steps). The second and subsequence dimensions separate proteins based on a physical property. For example, in some embodiments of the present invention proteins are separated by pI using isoelectric focusing (See e.g., Righetti, Laboratory Techniques in Biochemistry and Molecular Biology; Work, T. S.; Burdon, R. H., Elsevier: Amsterdam, p 10 [1983]). However, the present invention may employ any number of separation techniques including, but not limited to, ion exclusion, ion exchange, normal/reversed phase partition, size exclusion, ligand exchange, liquid/gel phase isoelectric focusing, and adsorption chromatography. In some embodiments (e.g., some automated embodiments), it is preferred that the separations be conducted in the liquid phase to enable products of the separation step to be fed directly into a subsequent liquid phase separation step.

In some embodiments, the proteins collected from the second or subsequent dimensions are identified using proteolytic enzymes, MALDI-TOF MS and MSFit database searching. Certain preferred embodiments are described in detail below. These illustrative examples are not intended to limit the scope of the invention. For example, although the examples are described using human samples, the methods and apparatuses of the present invention can be used with any desired protein samples including samples from plants and microorganisms.

Exemplary protein separation and analysis methods suitable for use with the present invention are described in more detail below. One skilled in the relevant arts recognizes that additional methods may be utilized. For example, additional protein separation and analysis methods are described in U.S. Patent applications 20040010126, 20020039747, 20050230315, 20040033591, 20040214233, 20020098595, 20030064527, and U.S. Pat. No. 6,931,325, each of which are incorporated herein by reference in their entireties.

In some preferred embodiments, lectin affinity is utilized as a first separation step to enrich for glycosylated proteins. Lectins are carbohydrates that bind to glycosylated proteins. The use of lectin affinity chromatography allows for a protein sample to be enriched in glycosylated proteins. The present invention is not limited to the use of lectin affinity chromatography for identifying glycosylation patterns. The present invention contemplates the use of any separation component that separates proteins based on the presence of, type of, or degree of glycosylation, including the use of other affinity columns that recognize sugars or carbohydrate structures.

Lectin affinity columns and chromatography medium are commercially available. For example, in one exemplary embodiment of the present invention, agarose bound lectins wheat Germ Agglutinin, Elderberry lectin, and Maackia amurensis lectin were purchased from Vector Laboratories (Burlingame, Calif., USA). However, the present invention is not limited to the lectin affinity resins described herein. Additional chromatography medium is commercially available. Candidate resins can be evaluated for their ability to bind serum glycoproteins using any suitable method including, but not limited to, those described herein. Protein samples are loaded onto the column and incubated to allow for binding. In some embodiments, non-specifically bound proteins are removed by washing the column with binding buffer. The captured glycoproteins are then released with an elution buffer.

In some embodiments, prior to lectin affinity chromatography, high abundance serum proteins are removed (e.g., using the ProtromeLab IgY-12 proteome partitioning kit (Beckman Coulter, Fullerton, Calif.)). This column enables removal of albumin, IgG, α1-antitrpsin, IgA, IgM, transferrin, haptoglobin, α1-acid glycoprotein, a2-macroglobin, HDL (apolipoproteins A-I and A-II) and fibrinogen in a single step. The present invention is not limited to a particular mechanism. Indeed, an understanding of the mechanism is not necessary to practice the present invention. Nonetheless, it is contemplated that the removal of high abundance serum proteins allows for the detection of low abundance proteins that may be masked in the presence of the high abundance proteins.

The following description provides certain embodiments for conducting separation on affinity purified glycosylated proteins according to the methods of the present invention. In some embodiments, affinity purified proteins are separated in one additional separation step. In other embodiments, two or more additional separation steps are utilized.

In some embodiments, the separation is isoelectric focusing (IEF). In some embodiments, IEF is performed in a buffer that is compatible with each of the subsequent steps in the separation/analysis methods. Although the present invention provides suitable buffers for use in the particular method configurations described below, one skilled in the art can determine the suitability of a buffer for any particular configuration by solubilizing protein sample in the buffer. If the buffer solubilizes the protein, the sample is run through the particular configuration of separation and detection methods desired. A positive result is achieved if the final step of the desired configuration produces detectable information (e.g., ions are detected in a mass spectrometry analysis). Alternately, the product of each step in the method can be analyzed to determine the presence of the desired product (e.g., determining whether protein elutes from the separation steps).

In some embodiments, n-octyl β-D-glucopyranoside (OGI, from Sigma) is used in the buffer. It is contemplated that detergents of the formula n-octyl SUGARpyranoside find use in these embodiments. The protein solution is loaded to a device that can separate the proteins according to their pI by isoelectric focusing. In some embodiments, the proteins are solubilized in a running buffer that is compatible with HPLC.

Three exemplary devices that may be used for this step are:

a) Rotofor

This device (Biorad) separates proteins in the liquid phase according to their pI (See e.g., Ayala et al, Appl. Biochem. Biotech. 69:11 [1998]). This device allows for high protein loading and rapid separations that require only four to six hours to perform. Proteins are harvested into liquid fractions after a 5-hour IEF separation. These liquid fractions are ready for analysis by HPLC. This device can be loaded with up to 1 g of protein.

b) Carrier Ampholyte Based Slab Gel IEF Separation with a Whole Gel Eluter

In this case the protein solution is loaded onto a slab gel and the proteins separate in to a series of gel-wide bands containing proteins of the same pI. These proteins are then harvested using a whole gel eluter (WGE, from Biorad). Proteins are then isolated in liquid fractions that are ready for analysis by HPLC. This type of gel can be loaded with up to 20 mg of protein.

c) IPG Slab Gel IEF Separation with a Whole Gel Eluter

Here the proteins are loaded onto a immobiline pI gradient slab gel and separated into a series of gel-wide bands containing proteins of the same pI. These proteins are electro-eluted using the WGE into liquid fractions that are ready for analysis by non-porous RP HPLC. The IPG gel can be loaded with at least 60 mg of protein.

In other embodiments, the separation is chromatofocusing. In chromatofocusing proteins are eluted from the column according to their pH, either one pH unit or fraction thereof, at a time. Columns for chromatofocusing are commercially available (e.g., Mono P HR 5/20 (Amersham Pharmacia, Uppsala, Sweden)). The column is equilibrated with a first buffer to define the upper pH range of the pH gradient. The proteins are then applied. The second focusing buffer is then applied to elute bound proteins, in the order of their isoelectric (pI) points. The pH of the second buffer is lower, and, defines the lower limit of the pH gradient. The pH gradient is formed as the eluting buffer titrates the buffering groups on the ion-exchanger.

In some embodiments, subsequent separation steps utilize HPLC (e.g., non-porous reverse phase HPLC). The novel combination of employing non-porous RP packing materials (Eichrom) with another RP HPLC compatible detergent (e.g., n-octyl 13-D-galactopyranoside) to facilitate the multi-phase separation is contemplated. This detergent is also compatible with mass spectrometry due to its low molecular weight. These columns are well suited to this task as the non-porous packing they contain provides optimal protein recovery and rapid efficient separations. It should be noted that there are many different low molecular weight non-ionic detergents that could be used for protein solubility while being compatible with RP HPLC. In some embodiments, the mobile phase contains a low level of a non-ionic low molecular weight detergent such as n-octyl β-D-glucopyranoside or n-octyl β-D-galactopyranoside as these detergents are compatible with RP HPLC and also with later mass spectrometry analyses (unlike many other detergents); the column should be held at a high temperature (around 60° C.); and the column should be packed with non-porous silica beads to eliminate problems of protein recovery associated with porous packings.

In some embodiments of the present invention, following separation, proteins are further characterized using mass spectrometry (e.g., following detection of a microarray). For example, in some embodiments, proteins are analyzed by mass spectrometry to determine their molecular weight and identity. The present invention is not limited by the nature of the mass spectrometry technique utilized for such analysis. For example, techniques that find use with the present invention include, but are not limited to, ion trap mass spectrometry, ion trap/time-of-flight mass spectrometry, time of flight/time of flight mass spectrometry, quadrupole and triple quadrupole mass spectrometry, Fourier Transform (ICR) mass spectrometry, and magnetic sector mass spectrometry. The following description of mass spectrometric analysis and 2-D protein display is illustrated with ESI or TOF mass spectrometry. Those skilled in the art will appreciate the applicability of other mass spectroscopic techniques to such methods.

For this purpose the proteins eluting from the separation can be analyzed simultaneously to determine molecular weight and identity. A fraction of the effluent is used to determine molecular weight by either MALDI-TOF-MS or ESI or TOF (LCT, Micromass) (See e.g., U.S. Pat. No. 6,002,127). The remainder of the eluent is used to determine the identity of the proteins via digestion of the proteins and analysis of the peptide mass map fingerprints by either MALDI-TOF-MS or ESI or TOF. The molecular weight 2-D protein map is matched to the appropriate digest fingerprint by correlating the molecular weight total ion chromatograms (TICs) with the UV-chromatograms and by calculation of the various delay times involved. The UV-chromatograms are automatically labeled with the digest fingerprint fraction number. The resulting molecular weight and digest mass fingerprint data can then be used to search for the protein identity via web-based programs like MSFit (UCSF).

In some embodiments, multiple mass spectrometry (e.g., 2, 3, or more) steps are utilized in the analysis of separated protein fractions. For example, in some embodiments, MALDI-MS/MS is utilized. In other embodiments, MS-MS is utilized.

In some embodiments, the data generated in the mass spectrometric analysis (e.g., TIC's or integrated and deconvoluted mass spectra) are converted to ASCII format and then plotted vertically, using a 256 step gray scale, such that peaks are represented as darkened bands against a white background.

In other embodiments, a color coded 1-D protein profile mass map is generated from differential display of protein molecular weights. In some embodiments, the image is displayed by a computer system as a color-coded mass map, where the intensity of the protein bands corresponds to colors of the rainbow, increasing from blue to green to yellow to red. Thus, the image provides a protein expression pattern that can be used to locate proteins that are differentially displayed in different samples (e.g., cells representing different stages of a cancer). Naturally, the image can be adjusted to show a more detailed zoom of a particular region or the more abundant protein signals can be allowed to saturate thereby showing a clearer image of the less abundant proteins. As the image is automatically digitized it may be readily stored and used to analyze the protein profile of the cells in question. Protein bands on the image can be hyper-linked to other experimental results, obtained via analysis of that band, such as peptide mass fingerprints and MSFit search results. Thus all information obtained about a given 1-D image, including detailed mass spectra, data analyses, and complementary experiments (e.g., immuno-affinity and peptide sequencing) can be accessed from the original image.

The data generated by the above-listed techniques may also be presented as a simple read-out. For example, when two or more samples are compared (e.g., cancerous and non-cancerous cells), the data presented may detail the difference or similarities between the samples (e.g., listing only the proteins that differ in identity or abundance between the samples). In this regard, when the differences between samples (e.g., cancerous and non-cancerous cells) are indicative of a given condition (e.g., cancer cell), the read-out may simply indicate the presence or identity of the condition. In one embodiment, the read-out is a simple +/− indication of the presence of particular proteins or expression patterns associated with a specific condition that is to be analyzed.

A useful feature of the liquid phase method of the present invention is the capability of the high resolution mass spectrometry to quantitate which allows the observer to record relative levels of each form of a given protein. Consequently, it is contemplated that one can determine the relative abundances of a given protein. In addition, post-translational modifications such as differing glycosylation patterns can be found. With a mass resolution of 5000 Da, a 50000 Da protein can be resolved from a 50010 Da protein. Quantitative comparison between 1-D images can be achieved by spiking samples with known amounts of standard proteins and normalizing images through landmark proteins. Thus, the observer can detect significant abundance changes in the protein profiles of different samples.

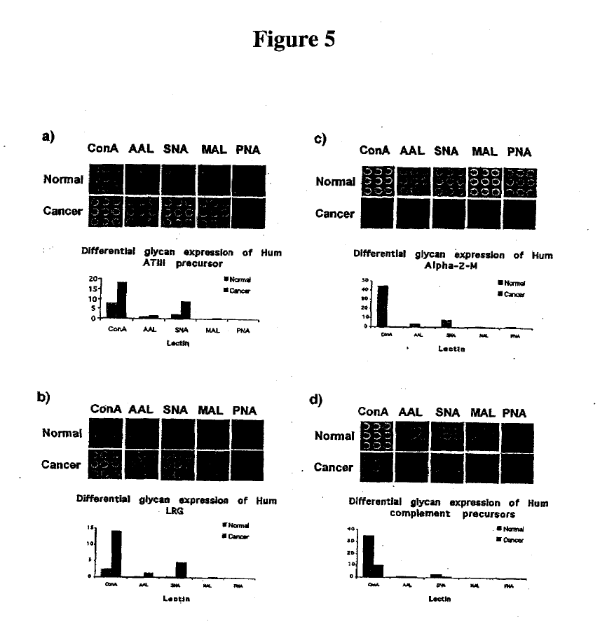

In some embodiments, the patterns of expression are expressed in relative fluorescence units as defined by the fluorescent moiety attached to the lectin. For example, as can be seen in FIG. 5, relative fluorescence of the ConA and SNA bound lectins to pancreatic cancer anti-thrombin III precursor glycoprotein is greater than the fluorescence seen for the other lectins being used as probes. FIG. 5 also demonstrates that the glycan structures which ConA and SNA bind to (Table 1) are more prevalent in pancreatic cancer serum glycoproteins than in non-cancer serum glycoproteins. In some preferred embodiments of the present invention, the information generated by the protein profile display is distributed in a coordinated and automated fashion. In some embodiments of the present invention, the data is generated, processed, and/or managed using electronic communications systems (e.g., Internet-based methods).

In some embodiments, a computer-based analysis program is used to translate the raw data generated by the protein profile map (e.g., identity and abundance of proteins in a sample) into data of predictive value for the clinician (e.g., the existence of a malignancy, the probability of pre-cancerous cells becoming malignant, or the type of malignancy). The clinician (e.g., family practitioner or oncologist) can access the predictive data using any suitable means. Thus, in some preferred embodiments, the present invention provides the further benefit that the clinician, who is not likely to be trained in molecular biology or biochemistry, need not understand the raw data of the protein profile map. The data is presented directly to the clinician in its most useful form. The clinician is then able to immediately utilize the information in order to optimize the care of the subject.

The present invention contemplates any method capable of receiving, processing, and transmitting the information to and from medical personal and subject. For example, in some embodiments of the present invention, a sample (e.g., a biopsy) is obtained from a subject and submitted to a protein profiling service (e.g., clinical lab at a medical facility, protein profiling business, etc.) to generate raw data. Once received by the protein profiling service, the sample is processed and a protein profile is produced (i.e., protein expression data), specific for the condition being assayed (e.g., presence of specific cancerous or pre-cancerous cells).

The protein profile data is then prepared in a format suitable for interpretation by a treating clinician. For example, rather than providing raw protein profile data, the prepared format may represent a risk assessment or probability of developing a malignancy that the clinician may use or as recommendations for particular treatment options (e.g., surgery, chemotherapy, or observation). The data may be displayed to the clinician by any suitable method. For example, in some embodiments, the protein profiling service generates a report that can be printed for the clinician (e.g., at the point of care) or displayed to the clinician on a computer monitor.

In some embodiments, the protein profile information (e.g., protein profile map) is first analyzed at a point of care or at a regional facility. The raw data is then sent to a central processing facility for further analysis. The central processing facility provides the advantage of privacy (all data is stored in a central facility with uniform security protocols), speed, and uniformity of data analysis. For example, using an electronic communication system, the central facility can provide data to the clinician, the subject, or researchers. The use of an electronic communications system allows protein profile data to be viewed by clinicians at any location. For example, protein profile data could be accessed by a specialist in the type of disease (e.g., cancer) that the subject is affected with. This allows even remotely located subjects to have their protein profiles analyzed by the leading experts in a particular field. The present invention thus provides a coordinated, timely, and cost effective system for obtaining, analyzing, and distributing life-saving information.

In some embodiments, all of the above described steps are automated, for example, into one discrete instrument. In one illustrative embodiment, the first dimension is lectin affinity chromatography, with the harvested liquid fractions being directly applied to the second dimension HPLC apparatus through the appropriate tubing. The products from the second dimension separation are then scanned and the data interpreted and displayed as a representation using the appropriate computer hardware and software. Alternately, the products from the second dimension fractions are sent through the appropriate microtubing to an on-plate MALDI digestion step, followed by mass spectrometry. The resulting data is received and interpreted by a processor. The output data represents any number of desired analyses including, but not limited to, identity of the proteins, mass of the proteins, mass of peptides from protein digests, dimensional displays of the proteins based on any of the detected physical criteria (e.g., size, charge, hydrophobicity, etc.), and the like. In preferred embodiments, the proteins samples are solubilized in a buffer that is compatible with each of the separation and analysis units of the apparatus. Using the automated systems of the present invention provides a protein analysis system that is an order of magnitude less expensive than analogous automation technology for use with 2-D gels (See e.g., Figeys and Aebersold, J. Biomech. Eng. 121:7 [1999]; Yates, J. Mass Spectrom., 33:1 [1998]; and Pinto et al., Electrophoresis 21:181 [2000]).

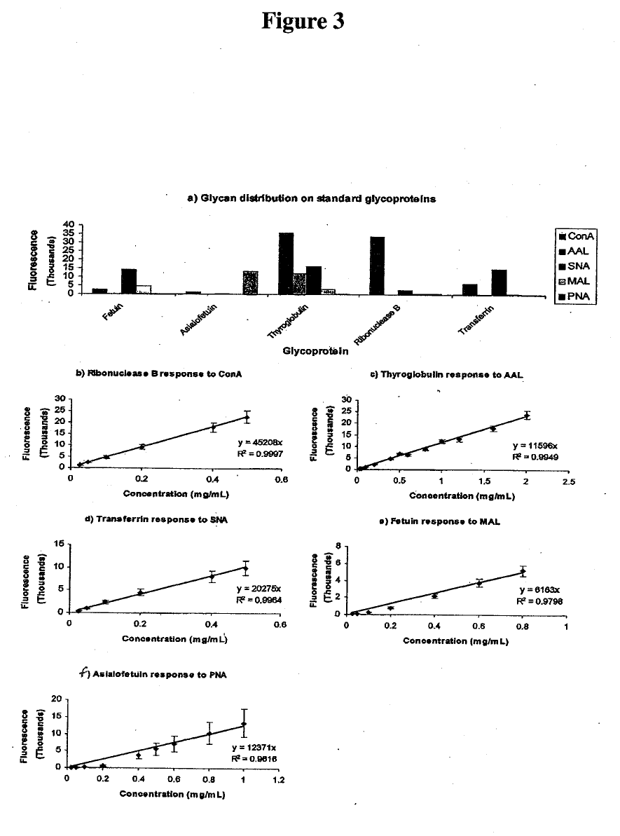

As described above, the separation techniques of the present invention were utilized to identify a series of glycoproteins and associated glycan structures. For example, FIG. 3 demonstrates the distribution of different glycan structures that are found on five different glycoproteins. The glycan structure of a-linked mannose is found in greater abundance on thyroglobulin and Ribonuclease B when compared to fetuin, asialofetuin, and transferrin, using ConA lectin (e.g., binds specifically to the a-linked mannose glycan structure). More importantly, FIG. 5 demonstrates the glycan structural differences between pancreatic cancer glycoproteins and non-cancer serum glycoproteins. Patterns in glycan structure within each of the four human glycoproteins are shown as defined by which labeled lectin probe binds to which glycoprotein, as well pattern of expression of those glycan structures found in pancreatic cancer compared to non-pancreatic cancer. In some embodiments, the present invention provides methods of diagnosing pancreatic cancer comprising assaying for the presence of such glycan structures. In preferred embodiments, serum is assayed for altered expression or glycosylation patterns. In other embodiments, tissue (e.g., biopsy tissue), urine, or blood is assayed.

The present invention is not limited to the glycosylated proteins or glycan structures listed. In some embodiments, additional glycan structures and glycosylated proteins are identified (e.g., using the methods of the present invention).

In some embodiments, the present invention provides methods for detection of expression of glycan structures in cancer (e.g., pancreatic cancer, prostate cancer, breast cancer, etc.). In some embodiments, expression is detected in tissue samples (e.g., biopsy tissue). In other embodiments, expression is detected in bodily fluids (e.g., including but not limited to, plasma, serum, whole blood, mucus, and urine). The present invention further provides panels and kits for the detection of glycan structures. In preferred embodiments, the presence of a glycan structure is used to provide a prognosis to a subject.

The present invention is not limited to the glycoproteins and glycan structures described above. Any suitable glycoprotein and glycan structure that correlates with cancer or the progression of cancer may be utilized, including but not limited to, those described in Tables 1 and 2. Additional glycoproteins and glycan structures are also contemplated to be within the scope of the present invention.

Any suitable method may be utilized to identify and characterize glycoproteins and glycan structures suitable for use in the methods of the present invention. In some embodiments, markers identified as being up or down-regulated in pancreatic cancer using the methods of the present invention are further characterized using gene expression microarray analysis, immunohistochemistry, Northern blot analysis, siRNA or antisense RNA inhibition, mutation analysis, investigation of expression with clinical outcome, as well as other methods disclosed herein. Differential glycosylation patterns may be detected by any method, including, but not limited to, mass spectroscopy, antibody affinity, chemical degradation and analysis, and the like.

In some embodiments, the present invention provides a panel for the analysis of a plurality of glycan structures. The panel allows for the simultaneous analysis of multiple glycan structures correlating with carcinogenesis and/or metastasis. For example, a panel may include two or more glycan structures identified as correlating with cancerous tissue, metastatic cancer, localized cancer that is likely to metastasize, pre-cancerous tissue that is likely to become cancerous, chronic pancreatitis, and pre-cancerous tissue that is not likely to become cancerous. Depending on the subject, panels may be analyzed alone or in combination in order to provide the best possible diagnosis and prognosis. Any of the glycan structures described herein may be used in combination with each other or with other known or later identified cancer glycan structures.

In other embodiments, the present invention provides an expression profile map comprising expression profiles of cancers of various stages or prognoses (e.g., likelihood of future metastasis). Such maps can be used for comparison with patient samples. Any suitable method may be utilized, including but not limited to, by computer comparison of digitized data. The comparison data is used to provide diagnoses and/or prognoses to patients.

In some embodiments, glycoproteins and glycan structures are detected by immunohistochemistry. In other embodiments, proteins are detected by their binding to an antibody that binds to a lectin of the present invention.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference is utilized.

In some embodiments, a computer-based analysis program is used to translate the raw data generated by the detection assay (e.g., the presence, absence, or amount of a given marker or markers) into data of predictive value for a clinician (See e.g., the above description of data analysis and distribution methods).

In some embodiments, the present invention provides kits for the detection and characterization of glycoproteins. In some embodiments, the kits contain one or more lectins for a cancer specific glycan structure, in addition to detection reagents and buffers. In some embodiments, the kits contain reagents for identifying glycosylated protein (e.g., the glycosylation detection reagents described above) in addition to reagents for identifying glycan structures. In some embodiments, the kits contain all of the components necessary and/or sufficient to perform at least one detection assay, including all controls, directions for performing assays, and any necessary or desired software for analysis and presentation of results.

In some embodiments, reagents (e.g., lectins) specific for the cancer markers of the present invention are fluorescently labeled. The labeled lectins are introduced into a subject (e.g., orally or parenterally). Fluorescently labeled lectins are detected using any suitable method (e.g., using the apparatus described in U.S. Pat. No. 6,198,107, herein incorporated by reference).

The present invention provides isolated antibodies. In preferred embodiments, the present invention provides monoclonal antibodies that specifically bind to an isolated polypeptide comprised of at least five amino acid residues of the lectin described herein. These antibodies find use in the diagnostic methods described herein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a cancer marker of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a cancer marker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against it is recovered from the immunized animal and the antibody is separated and purified.

As for the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a cancer marker of the present invention (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

### EXAMPLES

The following examples serve to illustrate certain preferred embodiments and aspects of the present invention and are not to be construed as limiting the scope thereof.

### Example 1

### Preparation of Experimental Samples

Experimental glycoprotein standards, fetuin from fetal calf serum, asialofetuin from fetal calf serum, porcine thyroglobulin, bovine ribonuclease B, α-acid glycoprotein and human transferrin were purchased from Sigma Corporation (St. Louis, Mo.). A 20 mg/mL stock solution of each standard was made by dissolving standards in de-ionized water. A dilution series was made for each of the standard glycoproteins, yielding the final concentrations of 2, 1.6, 1.2, 1, 0.8, 0.6, 0.5, 0.4, 0.2, 0.1, 0.05, and 0.025 mg/mL. The dilutions were made directly into printing buffer (65 mM Tris-HCl, 1% SDS, 5% dithiothreitol (DTT) and 1% glycerol) to avoid drying and reconstitution to minimize sample loss.

Human normal serum, pancreatitis serum and pancreatic cancer serum were provided by University Hospital (University of Michigan, Ann Arbor, Mich.). Forty milliliters of blood (Vacutainer® red top tubes with no anticoagulant) was provided by each patient. The samples were permitted to sit at room temperature (RT) for a minimum of 30 min (and a maximum of 60 min) to allow clot formation, and then the tubes were centrifuged at 1,300×g at 4° C. for 20 min. The serum was transferred to a polypropylene capped tube and stored at −70° C. until assayed.

### Example 2

### Lectin Affinity Glycoprotein Extraction

The same procedure as described below was performed for each serum sample. Agarose bound lectin (Wheat Germ Agglutinin (WGA)) was purchased from Vector Laboratories (Burlingame, Calif., USA). The WGA was packed into disposable screw end-cap spin column with filters at both ends. The column was first washed with 500 μl binding buffer (20 mM Tris, 0.15 M NaCl, pH 7.4) by centrifuging the spin columns at 500 rpm for 2 min. A protease inhibitor stock solution was prepared by dissolving one complete EDTA-free Protease inhibitor cocktail tablet (Roche, Indianapolis, Ind.) in 1 ml water. The stock solution was added to the binding buffer and elution buffer (0.5 M N-acetyl-glucosamine in 20 mM Tris and 0.5 M NaCl, pH 7.0) at a ratio of (v/v) 1:50. Fifty μl of a serum sample was diluted with 500 μl binding buffer and loaded onto a column and incubated for 15 min. The column was centrifuged for 2 min at 500 rpm to remove the non-binding fraction and washed twice with 600 μl binding buffer. The captured glycoproteins were released with 150 μl elution buffer and collected by centrifugation at 500 rpm for 2 min. This step was repeated twice and the eluted fractions were pooled.

### Example 3

### HPLC Separation of Lectin-Bound Glycoproteins

The same procedure as described below was performed for each serum sample. The enriched glycoprotein fraction was loaded onto a nonporous silica reverse phase high-performance liquid chromatography (NPS-RP-HPLC) column for separation. High separation efficiency was achieved by using an ODSIII-E (4.6×33 mm) column (Eprogen, Inc., Darien, Ill.) packed with 1.5 μm non-porous silica. To collect purified proteins from NPS-RP-HPLC, the reversed-phase separation was performed at 0.5 mL/min and monitored at 214 nm using a Beckman 166 Model UV detector (Beckman-Coulter). Proteins eluting from the column were collected using an automated fraction collector (Model SC 100; Beckman-coulter) controlled by an in-house designed DOS-based software program. To enhance the speed, resolution, and reproducibility of the separation, the reversed phase column was heated to 60° C. by a column heater (Jones Chromatography, Model 7971). Both mobile phase A (water) and B (ACN) contained 0.1% v/v TFA. The gradient profile used was as follows: 5% to 15% B in 1 min, 15% to 25% B in 2 min, 25% to 30% B in 3 min, 30% to 41% B in 15 min, 41% to 47% B in 4 min, 47% to 67% B in 5 min and 67% to 100% B in 2 min.

### Example 4

### Production and Probing of Glycoprotein Microarrays

Purified and separated serum sample glycoproteins, or glycoprotein standards (Example 1), were printed on nitrocellulose slides (Whatman Schleicher & Schuell BioScience, Keene, N.H.) using a non-contact printer, Nanoplotter 2.0 (GeSiM, Germany). Prior to printing, the proteins were dried down in a 96-well plate and resuspended in 15 μL of printing buffer with stirring overnight at 4° C. Each spotting event resulted in approximately 500 pL of sample being deposited by a piezoelectric mechanism. The event was programmed to occur 5 times per spot to ensure that approximately 2.5 nL per sample was being spotted. Each sample was further spotted as nine replicates. The resulting spots were approximately 450 μm in diameter and the spacing between spots was maintained at 600 μm. After printing, the slides were allowed to dry for 24 hours.

Blocking was achieved by incubating the slides with 1% bovine serum albumin (BSA) and 0.1% Tween-20 in 1× phosphate buffered saline (PBS) overnight. Blocked slides were probed with biotinylated lectin in a solution of PBS-T (0.1% Tween 20 in 1×PBS). The lectins used in the study were biotinylated Peanut Agglutinin (PNA), Sambucus Nigra bark lectin (SNA), Aleuria Aurentia (AAL), Concanavalin A (ConA) and Maackia Amurensis lectin II (MAL), all purchased from Vector Laboratories (Burlingame, Calif., USA). The working concentration of all lectins used was 5 μg/mL except for SNA, which was used at 10 μg/mL as per vendor recommendation. After the primary probe, all slides were washed with PBS-T 5 times for 5 min each. The secondary probe was performed with a streptavidin-AlexaFluor®555 conjugate (Invitrogen, Carlsbad, Calif.) in a working concentration of 1 μg/mL containing 0.5% BSA, 0.1% Tween-20 in 1×PBS. After the secondary probe, the slides were washed 5 times for 5 minutes each in PBS-T and completely dried using a high-speed centrifuge (Thermo Electron Corp., Milford, Mass.). The dried slides were scanned using an Axon 4000A scanner in the green channel and GenePix® Pro 3.0 software (Molecular Devices, Sunnyvale, Calif.) was used for data acquisition and analysis.

### Example 5

### Protein Digestion by Trypsin

Fractions obtained from NPS-RP-HPLC were concentrated down to approximately 20 μL using a SpeedVac concentrator (Thermo, Milford, Mass.) operating at 45° C. Twenty μl of 100 mM ammonium bicarbonate (Sigma) were mixed with each concentrated sample to obtain a pH value of approximately 7.8. TPCK modified sequencing grade porcine trypsin (0.5 μl, Promega, Madison, Wis.) was added to the samples which were then vortexed prior to a 12-16 hour incubation on a 37° C. agitator.

### Example 6

### Glycan Cleavage by PNGase F and Glycan Purification

For glycan cleavage and purification, glycoproteins were dried down completely and redissolved in 40 μl 0.1% (w/v) RapiGest solution (Waters, Milford, Mass.) prepared in 50 mM NH₄HCO₃ buffer, pH 7.9 to denature the protein. Protein samples were reduced with 5 mM DTT for 45 min at 56° C. and alkylated with 15 mM iodoacetamide in the dark for 1 h at room temperature. Two μl of enzyme PNGase F (QA-Bio, Palm Desert, Calif.) was added to the samples and the solutions were incubated for 14 h at 37° C. Released glycans were purified using SPE micro-elution plates (Waters) packed with HILIC sorbent (5 mg). The micro-elution SPE device was operated using a centrifuge with a plate adaptor (Thermo). Protein and detergent were removed during this step. Glycans were further purified using a graphitized carbon cartridge (Alltech, DeerWeld, Ill.) to remove salt. 25% ACN with 0.05% TFA was used to elute the carbohydrates.

### Example 7

### Protein Identification by Mass Spectrometry LC-MS/MS

Digested peptide mixtures from non-porous substrate RP HPLC collection were separated by a capillary RP column (C18, 0.3×150 mm) (Michrom Biosciences, Auburn, Calif.) on a Paradigm MG4 micro-pump (Michrom Biosciences) with a flow rate of 5 μl/min. The gradient started at 5% ACN, was ramped to 60% ACN in 25 min and finally ramped to 90% in another 5 min. Both solvent A (water) and B (ACN) contain 0.1% formic acid. The resolved peptides were analyzed on an LTQ mass spectrometer with an ESI ion source (Thermo, San Jose, Calif.). The capillary temperature was set at 175° C., spray voltage was 4.2 kV and capillary voltage was 30 V. The normalized collision energy was set at 35% for MS/MS. MS/MS spectra were searched using the SEQUEST algorithm incorporated in Bioworks software (Thermo) against the Swiss-Prot human protein database. One mis-cleavage was allowed during the database search. Protein identification was considered positive for a peptide with X_{corr} of greater than or equal to 3.0 for triply-, 2.5 for doubly- and 1.9 for singly charged ions.

MS and MS² spectra of glycan samples were acquired on a Shimadzu Axima QIT MALDI quadrupole ion trap-ToF (MALDI-QIT) (Manchester, UK). Acquisition and data processing were controlled by Launch-pad software (Karatos, Manchester, UK). A pulsed N₂ laser light (337 nm) with a pulse rate of 5 Hz was used for ionization. Each profile resulted from 2 laser shots. Argon was used as the collision gas for CID and helium was used for cooling the trapped ions. The TOF was externally calibrated using 500 fmol/ul of bradykinin fragment 1-7 (757.40 m/z), angiotensin II (1046.54 m/z), P14R(1533.86 m/z), and ACTH(2465.20 m/z) (sigma). 25 mg/ml 2,5-dihydroxybenzonic acid (DHB) (LaserBio Labs, France) was prepared in 50% ACN with 0.1% TFA. 0.5 μl glycan sample was spotted on the stainless-steel target and 0.5 μl matrix solution was added followed by air drying.

### Experimental Results

To determine the feasibility of using a glycoprotein microarray to study separated pre-purified glycoproteins, initial studies were done using standards with known glycan structures in order to assess the specificities of the lectins used. Six standard glycoproteins were used to assess the feasibility of a glycoprotein microarray strategy as described in Example 1. Table 1 describes the binding specificities of the biotinylated lectins used for glycan detection. ConA recognizes α-linked mannose including high mannose-type and mannose core structures. Both MAL and SNA recognize sialic acid on the terminal branches, while SNA binds preferentially to sialic acid attached to terminal galactose in an (α2,6) and to a lesser degree, an (α-2,3) linkage. MAL could detect glycans containing NeuAc-Gal-GlcNac with sialic acid at the 3 position of galactose. In contrast, PNA binds de-sialylated exposed galactosyl (β-1,3) N-acetylgalactosamine. In fact, sialic acid in close proximity to the PNA receptor sequence will inhibit its binding. AAL recognizes fucose linked (α-1,6) to N-acetylglucosamine or to fucose linked (α-1,3) to N-acetyllactosamine. The combination of these five lectins can cover a majority of N-glycan types reported and differentiate them according to their specific structures.

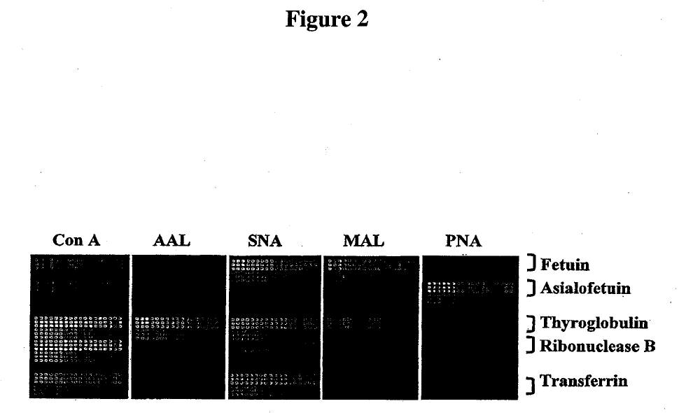

Printed glycoprotein standards were incubated with biotinylated lectins and assayed for binding. The bound biotinylated lectins were subsequently detected with streptavidin conjugated to AlexaFluor555. This sandwich-type detection scheme was employed because the very specific biotin-streptavidin interaction should improve signal to noise ratio significantly. FIG. 2 shows the images obtained when slides were probed with each of the lectins. Background fluorescence was at a minimum with the processing conditions used. Data illustrated in FIG. 3a supports what is known of glycan distribution on the standard glycoproteins. The abundant glycan structures of bovine fetuin are sialylated, bi- and tri-antennary complex-type N-glycans (core non-fucosylated). The sialic acid residues are found in both (α-2,3) and (α-2,6) linkages. Abundant glycans in asialofetuin include asialo-bi and asialo-tri antennary N-linked oligosaccharides. Dominant porcine thyroglobulin glycans include disialylated biantennary N-linked oligosaccharides with core fucose and oligomannose N-linked oligosaccharide with 5-9 mannosyl residues. The glycan of ribonuclease B is mannose type i.e. Man₅₋₉GlcNac₂. The dominant glycan in transferrin is sialylated, biantennary complex-type N-glycan.

As shown in FIG. 3a, Con A binds strongly to thyroglobulin and ribonuclease B since both of their glycans contain oligomannose N-linked oligosaccharide with 5-9 mannosyl residues. Transferrin, fetuin and asialofetuin bind weakly to Con A as mannose residues are only present in their core structure and not in the exposed branches. SNA bound to fetuin, thyroglobulin and transferrin, which have all been reported to possess sialic acid moieties on their glycans, while MAL only bound to Fetuin and porcine thyroglobulin, which have sialic acid attached in an (α-2,6) position to a noticeable extent. These two lectins can therefore be used to discriminate between sialic acid residues in an (α-2,3) vs (α-2,6) linkage due to the more specific interaction of MAL.

This data demonstrate the importance of using multiple lectin detection schemes in microarray formats for explicit differentiation of glycan structures. PNA bound to only asialofetuin since it is the only standard used that has de-sialylated, exposed galactosyl (β-1,3) N-acetylgalactosamine residues in its glycan structure. This lectin was also found to be the most specific lectin used. As shown in FIGS. 2 and 3a, AAL binds strongly to porcine thyroglobulin which is the only standard used whose main structure consists of disialylate, biantennary N-linked oligosaccharide with core fucose.

In all cases where standard proteins elicited response, the limit of detection was found to be between a concentration of 0.05-0.1 mg/mL. This corresponds to an absolute protein content of between 125 pg to 250 pg. On average, glycoproteins fall in the molecular weight range of about 50 kDa. Consequently, 125-250 pg translates into a 2.5 to 5 fmols detection limit. Mass spectrometric glycan structure determination often requires higher amounts of sample due to the need for multiple sample handling steps as well as MSⁿ fragmentation requirements for complete structural information. In the case of MAL where only fetuin was found to bind, the limit of detection was much higher at almost 1 mg/mL protein concentration corresponding to 2.5 ng or 50 fmol total protein content. If the printing buffer composition is changed so that spots spread out to a lesser degree across the array surface, the density of sample per spot area could be increased, resulting in lower limits of detection.

To determine the linearity of response to individual lectins for each of the standard protein, curves were generated based on the fluorescence response of all printed spots and their replicates (FIGS. 3b-f). In addition to the 9 replicates on each slide, data points were collected from two processed slides for each lectin in order to assess the variability between slide images processed in the same manner and on the same day. It was found that all proteins showed a linear response to each of the lectins within a 0.025-1 mg/mL concentration range (Figures b-f). However linearity of response was optimal in a range of 0.025-0.5 mg/mL.

All standard curves were unique to the standard protein that was being used to generate it. This is consistent with the fact that a lectin does not measure quantity of a protein spotted but reflects the extent to which a particular glycan structure is expressed on that protein. To illustrate this, the dominant glycan structures on Ribonuclease B and Transferrin was determined by tandem mass spectrometry. Based on mass spectrometry, ribonuclease B has a mannose-rich glycan structure not present in transferrin. This explains FIG. 3A where even at the same concentration of standards, ribonuclease B responds to ConA to a much greater degree than transferrin. Using a glycoprotein microarray strategy together with mass spectrometry oftentimes yields a more complete means to characterize glycan structures on proteins. Therefore, it is demonstrated that glycoprotein microarrays can be used to study differences in glycosylation states of individual proteins in more complex biological samples.

Enriched and pre-fractionate glycoproteins from human serum was used in glycoprotein microarrays to see if differences were evident in sera from biologically distinct states. As illustrated in FIG. 1, serum was first purified for glycoproteins using Wheat Germ Agglutinin (WGA). WGA can bind oligosaccharides containing terminal N-acetylglucosamine or chitobiose as well as sialic acid residues, structures that are common to many serum and membrane glycoproteins. The purified and enriched glycoproteins were then separated in a second dimension by non-porous reverse phase HPLC. This separation resolved the enriched glycoproteins into approximately 30 fractions. When 2.5 mg (˜50 μL raw serum) serum proteins were enriched, approximately 100 μg of glycoproteins were typically recovered. Only half of this sample was run in the second dimension. After considering recovery from the reverse phase column and the number of fractions collected in the second dimension, it can be estimated that each fraction contained an average of 1-2 μg of protein (this amount is proportional to the height of relative peaks). All collected fractions were dried down and resuspended in 15 μL of printing buffer so that the working concentrations of the glycoproteins printed were in the range of 0.1-0.2 mg/mL. This range falls between the concentrations that were used for the standard glycoproteins ensuring similarity in parameters used in both studies.

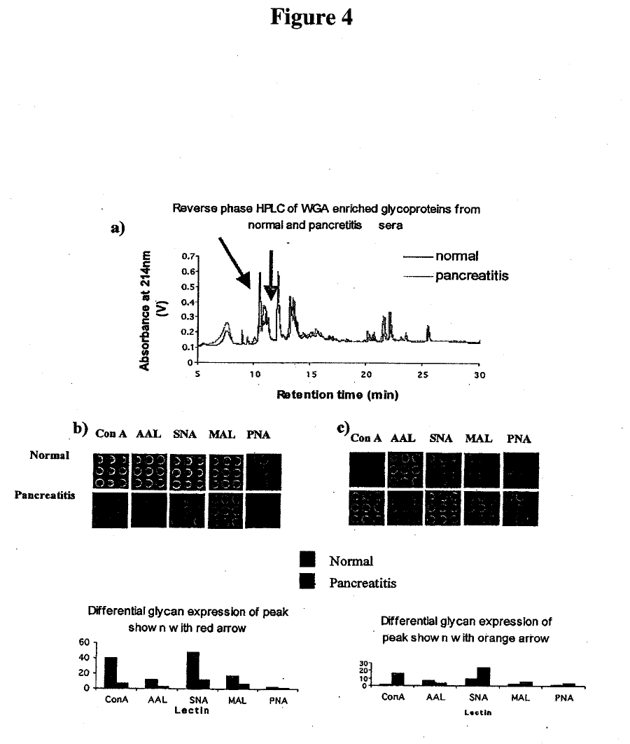

To access changes in glycosylation patterns between sera from different biological states, WGA enriched glycoproteins from normal and pancreatitis serum were fractionated and spotted on nitrocellulose slides. The reverse-phase chromatogram of enriched glycoproteins from the two sera samples showed some differences in peak heights. In addition to confirming the concentration difference shown by the different peak heights, the glycoprotein microarray also indicated different glycosylation pattern for the observed differences. FIG. 4 shows the reverse phase chromatogram demonstrating differences between the two samples. Based on the peak heights alone, the peak on the left (left arrow) is 2 to 3 times over-expressed in normal serum compared to pancreatitis serum (right arrow). Microarray data in FIG. 4 indicated that response to some of the lectins for the same peak is often more than 2 to 3 times in the normal serum compared to pancreatitis serum. This suggests that the protein is more glycosylated in normal serum particularly in mannose and fucosylated moieties because response to ConA and AAL was approximately 5 and 6 times higher respectively in the normal serum sample compared to pancreatitis serum. Additionally, the peak associated with the right arrow (pancreatitis serum) showed another interesting trend. Although the peak height was less than two times higher in the pancreatitis serum compared to the normal serum, response to AAL was higher in the normal sample. This suggests that the protein concerned is much less fucosylated in chronic pancreatitis. Furthermore, the protein showed a higher expression of mannose on its glycans since response to ConA was 10 times higher in pancreatitis serum compared to normal serum (FIG. 4c).

Experiments were performed to access the differences in enriched glycoproteins from normal versus pancreatic cancer sera. Pancreatic cancer is currently difficult to diagnose at an early stage due to lack of early diagnostic markers and due to similarity to pancreatitis in early stages of the disease. More differences were observed between normal and pancreatic cancer sera than between normal and pancreatitis sera. FIG. 5 shows sections of arrays comparing normal and pancreatic cancer serum glycoproteins. In all data shown, reverse-phase chromatograms indicated similar protein amounts since peak heights and widths were comparable. It can be seen from the bar graphs that sialic acid was more abundant in selected cancer serum glycoproteins compared to normal serum glycoproteins (FIGS. 5a and 5b). Conversely, some peaks showed higher mannosylation in normal serum compared to cancer serum (FIGS. 5c and 5d). More fucosylation was seen in cancer serum fractions compared to normal serum fractions. But the extent of differential expression appeared to be much less than the sialylation. The proteins illustrated in FIG. 5 were identified by tandem mass spectrometry and are presented in Table 2.

TABLE 2
Protein IDs of microarray data comparisons shown in
FIG. 5 with reported glycosylation site references.

| Differential | |
|---|---|
| glycosylation | |
| data shown | Protein ID |
| FIG. 5a | ANT3_HUMAN P01008 Antithrombin-III precursor |
| | (ATIII). |
| FIG. 5b | A2GL_HUMAN P02750 Leucine-rich alpha-2- |
| | glycoprotein precursor (LRG). |
| | HEP2_HUMAN P05546 Heparin cofactor II precursor |
| | (HC-II) (Protease inhibitor leuserpin 2) (HLS2). |
| FIG. 5c | A2MG_HUMAN P01023 Alpha-2- |
| | macroglobulin precursor (Alpha-2-M). |
| FIG. 5d | CO3_HUMAN P01024 Complement C3 precursor |
| | CO4_HUMAN P01028 Complement C4 precursor |

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A method for high throughput determination of glycan structures comprising:
a) providing a sample comprising a glycoproteome of a biological sample,
b) providing a solid support,
c) applying said sample to said solid support such that discrete areas containing said sample are created on said solid support,
d) providing one or more lectins,
e) contacting said one or more lectins with said solid support containing said discrete areas containing said sample, and
f) determining the glycan structure of glycoproteins in said glycoproteome by the binding of said one or more lectins to said discrete areas on said solid support containing said sample.

2. The method of claim 1, further comprising determining the presence or absence of cancer based upon said determining the glycan structure of glycoproteins in said glycoproteome by the binding of said one or more lectins to said discrete areas on said solid support containing said sample.

3. The method of claim 2, wherein the presence or absence of cancer is the presence or absence of pancreatic cancer.

4. The method of claim 1, wherein said one or more lectins are conjugated to a first member of a binding pair.

5. The method of claim 4, further comprising a second binding member of said binding pair that binds to said one or more lectins.

6. The method of claim 5, wherein said first or second member of said binding pair is a fluorescent moiety.

7. The method of claim 5, wherein said second binding member comprises streptavidin.

8. The method of claim 7, wherein said streptavidin molecule further comprises a fluorescent moiety.

9. The method of claim 1, wherein said glycoproteome sample is derived from serum.

10. The method of claim 9, wherein said glycoproteome serum sample is from a group consisting of normal, pancreatitis, or pancreatic cancer serum.

11. The method of claim 1, wherein said determining the glycan structure for said glycoproteins in said glycoproteome is further used to determine the presence or absence of cancer.

12. The method of claim 1, wherein said glycoproteome sample is initially purified on a lectin column.

13. The method of claim 12, wherein said initially purified sample is further separated and fractionated using non-porous reverse phase HPLC.

14. The method of claim 1, wherein said one or more lectins comprises two or more lectins.

15. The method of claim 1, wherein said one or more lectins comprises three or more lectins.

16. The method of claim 1, wherein said one or more lectins comprises four or more lectins.

17. The method of claim 14, wherein said two or more lectins is selected from the list consisting of Concanavalin A, Maackia amurensis II, Aleuria aurantia, Sambucus nigra bark, and Peanut agglutinin.

18. The method of claim 15, wherein said three or more lectins is selected from the list consisting of Concanavalin A, Maackia amurensis II, Aleuria aurantia, Sambucus nigra bark, and Peanut agglutinin.

19. The method of claim 16, wherein said four or more lectins is selected from the list consisting of Concanavalin A, Maackia amurensis II, Aleuria aurantia, Sambucus nigra bark, and Peanut agglutinin.

20. A composition comprising a solid surface comprising discrete areas upon which are affixed purified or partially purified glycoproteins, one or more lectins wherein a lectin recognizes a different glycan structure, and a compound which binds to said lectin either directly or indirectly.

21. The composition of claim 20, wherein said one or more lectins are conjugated to a first member of a binding pair.

22. The composition of claim 21, wherein said first binding pair member is biotin.

23. The compound of claim 20, wherein said compound that binds directly to said one or more lectins is a fluorescently labeled antibody.

24. The composition of claim 20, wherein said compound that binds indirectly to said one or more lectins is a fluorescently labeled streptavidin molecule.
